(19) United States
(12) Patent Application Publication
(10) Pub. No.: US 2002/0128522 A1
(43) Pub. Date: Sep. 12, 2002

(54) Process for the preparation of 4,4' -dihalogen-o-hydroxydiphenyl compounds

(76) Inventors: Armando Di Teodoro, Rheinfelden (DE); Werner Holzl, Eschentzwiller (FR); Dieter Reinehr, Kandern (DE); Rudolf Zink, Therwil (CH)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/137,791

(22) Filed: May 2, 2002

Related U.S. Application Data
(63) Continuation of application No. 09/806,359, filed on Mar. 29, 2001, now abandoned.
Application No. 09/806,359 is a 371 of international application No. PCT/EP99/07158, filed on Sep. 27, 1999.

(30) Foreign Application Priority Data
Oct. 6, 1998 (EP) 98811002.9

(51) Int. Cl. C07C 43/20

(52) U.S. Cl. 568/637

(57) ABSTRACT

Disclosed is a four-step process for the preparation of 4,4′-dihalogen-o-hydroxydiphenyl compounds of formula

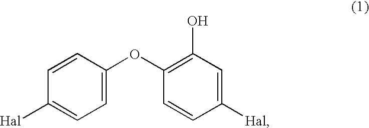

by halogenation of a diphenyl compound (step 1a) or by the reaction of p-halophenol and p-dihalobenzene (step 1b), acylation of the dihalogen compound in a Friedel-Crafts reaction (2^{nd} step), oxidation of the acyl compound (3^{rd} step) and hydrolysis (4^{th} step).

The compounds of formula (1) are used for protecting organic materials and objects from microorganisms.

The present invention relates to a process for the preparation of 4,4′-dihalogen-o-hydroxy-diphenyl compounds of formula

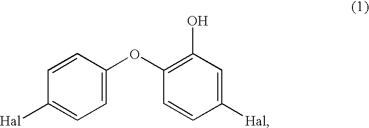

wherein

Hal is a halogen atom,

and to the use of these compounds for protecting organic materials and objects from micro-organisms.

The preparation of 4,4′-dihalogen-o-hydroxydiphenyl compounds is usually carried out by diazotisation and subsequent hydrolysis of 2-amino-4,4′-dichlorodiphenyl ether (compound of formula (2)):

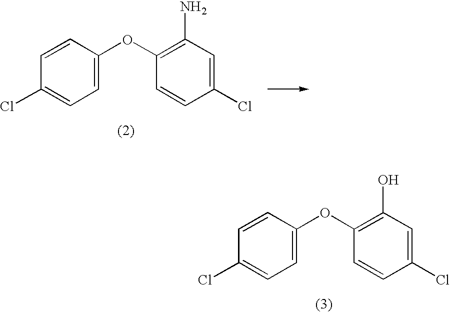

However, the yield obtained by this method of preparation is unsatisfactory as different chemical reactions may take place concurrently.

Accordingly, this invention has for its object to provide an economic process for the preparation of 4,4′-dihalogen-o-hydroxydiphenyl compounds in which undesirable concurrent reactions are suppressed.

This object is achieved in accordance with this invention by a four-step reaction, where in the first step a diphenyl compound is halogenised or p-halophenol is reacted with p-dihalophenol in the presence of copper and/or copper salts; in the second step the dihalogen compound is acylated in a Friedel-Crafts reaction; in a third step the acyl compound is oxidised, and in a fourth step the oxidised compound is hydrolysed, corresponding to the following reaction scheme:

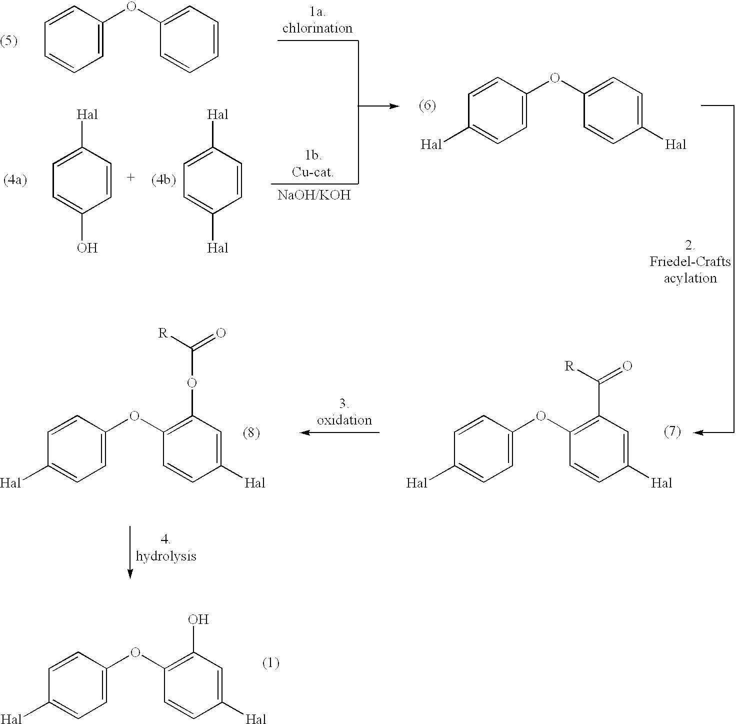

In the above scheme:

R is C₁-C₈alkyl which is unsubstituted or substituted by 1 to 3 halogen atoms or hydroxy; unsubstituted C₆-C₁₂aryl; or C₆-C₁₂aryl which is substituted by 1 to 3 halogen atoms, C₁-C₅alkyl or C₁-C₈alkoxy, or their combinations; and

Hal is a halogen atom;

C₁-C₈alkyl is branched or unbranched alkyl, for example methyl, ethyl, propyl, isopropyl, n-butyl, sec-butyl, isobutyl, t-butyl, 2-ethylbutyl, n-pentyl, isopentyl, 1-methylpentyl, 1,3-di-methylbutyl, n-hexyl, 1 -methylhexyl, n-heptyl, isoheptyl, 1,1,3,3-tetramethylbutyl, 1 -methyl-heptyl, 3-methylheptyl, 2-ethylhexyl or n octyl.

C₁-C₈Alkoxy is straight-chain or branched radicals, for example methoxy, ethoxy, propoxy, butoxy, pentyloxy, hexyloxy, heptyloxy or octyloxy.

Halogen is fluoro, bromo or, preferably, chloro.

In formulae (6) and (7) of the above reaction scheme, R is C₁-C₄alkyl and, preferably, methyl.

If halogen is preferably chlorine, the chlorination agent used for reaction step 1a is, for example, sulfuryl chloride or, preferably, gaseous chlorine. The reaction is preferably carried out in the presence of a catalyst, such as dibenzothiophene, methyl sulfide, propyl sulfide, phenyl sulfide, a Lewis acid, such as aluminium chloride, or mixtures of these compounds. A particularly suitable catalyst for the novel chlorination reaction is a mixture of propyl sulfide and an equimolar amount of aluminium chloride.

The temperature for the reaction of the first step can be chosen from within a wide range, for example from −10 to 50° C. The reaction is preferably carried out at a temperature from 0 to 40° C.

The reaction times are also within a wide range. The reaction is normally carried out over 1 to 48 hours, preferably over 2.5 to 10 hours.

The reaction of step 1b is usually carried out at temperatures from 120 to 200, preferably from 130 to 170° C, it being necessary for the phenol compound of formula (4a) and the dihalogen compound of formula (4b) to be present in a stoichiometric ratio, and the alkali hydroxide must be present in less than equivalent amount (20 to 80% of theory).

The copper catalysts used are preferably the copper salts conventionally used for the Ullmann synthesis, for example copper(II) oxide, copper(I) oxide, copper carbonate, basic copper carbonate, copper(I) chloride, copper(II) chloride, copper(I) bromide, copper(II) bromide or copper sulfate.

Further details on the reaction step 1b are to be found in DE-OS-2,242,519.

The acylation reaction (2^{nd} step) is usually carried out in the presence of a Lewis acid, such as aluminium chloride. The Lewis acid is used in this case in 1 to 3, preferably 1.25 to 2, molar amount, based on the halogenated acyl compound of formula (5). A suitable acylation reagent for this reaction is an acyl halide, preferably acetyl chloride. Other suitable acylation

agents are, for example,

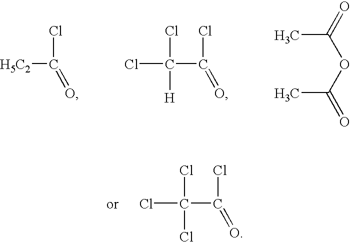

In this instance, Lewis acid and acylation reagent are preferably used in equimolar amounts. The reaction is carried out in the solvents conventionally used for Friedel-Crafts reactions, such as halogenated solvents, preferably methylene chloride or ethylene chloride.

In a special embodiment of this invention, the acylation reaction is carried out in the presence of acetyl chloride and aluminium chloride, which are used in equimolar amounts.

The reaction time is of subordinate importance for this reaction step and may vary within a wide range, for example from 1 to 18 hours.

The halogenation reaction (first step) and the acylation reaction (second step) as well as the optionally repeated chlorination reaction are preferably carried out in the same reaction vessel and are thus one-pot reactions.

The oxidation of the acyl compound of formula (6) to the compound of formula (7) (Baeyer-Villiger oxidation) can be carried out with different oxidants. Suitable oxidants are for example:

equimolar mixture of dilute peracetic acid and acetic anhydride in the presence of a catalytic amount of perchloric acid;

excess of 3-chloro-perbenzoic acid in water;

di-peroxydodecane diacid (DPDDA);

mixture of dilute peracetic acid and acetic anhydride and sulfuric acid;

persulfuric acid;

mixture of concentrated sulfuric acid, anhydrous acetic acid and hydrogen peroxide;

mixture of m-chloroperbenzoic acid (MCPBA), trifluoroacetic acid and dichloromethane;

mixture of sodium perborate and trifluoroacetic acid;

mixture of formic acid, hydrogen peroxide, acetic anhydride, phosphoropentoxide and acetic acid;

mixture of acetic acid, hydrogen peroxide, acetic anhydride and phosphoropentoxide;

mixture of K₂S₂O₈, sulfuric acid and a 1:1 -water/methanol mixture;

mixture of acetic acid and the potassium salt of monoperoxomaleic acid;

mixture of trichloromethylene, the potassium salt of monoperoxomaleic acid and sodium hydrogen sulfate;

mixture of maleic anhydride, acetic anhydride, hydrogen peroxide and trichloromethane;

mixture of maleic anhydride, a urea-hydrogen peroxide complex and acetic acid;

Mg-monoperphthalate.

A mixture of concentrated sulfuric acid, anhydrous acetic acid and hydrogen peroxide is preferably used for the oxidation.

Where required, a commercially available wetting agent may be added to the oxidant.

The reaction times may be within a wide range from about 1 to about 24 hours, preferably from 1 to 5 hours.

The reaction temperature ranges from −20° C. to about 80° C. The reaction is preferably carried out at room temperature.

The subsequent hydrolysis to the desired 4,4′-dihalogen-o-hydroxydiphenyl ether of formula (1) proceeds quantitatively.

The 4,4′-dihalogen-o-hydroxydiphenyl compounds prepared according to this invention are insoluble in water, but are soluble in dilute sodium hydroxide solution and potassium hydroxide solution and in virtually all organic solvents. Thanks to these solubility preconditions they have very versatile applicability for fighting microorganisms, in particular bacteria, and for protecting organic materials and objects from microorganisms. They are thus particularly suitable for the disinfection, deodorisation and the general antimicrobial treatment of the skin, mucosae and all integumentary appendages (hair), very particularly for the disinfection of hands and wounds.

They are therefore suitable as an antimicrobial active substance in body-care products, for example shampoos, bath additives, hair-care products, liquid and solid soaps (based on synthetic surfactants and salts of saturated and/or unsaturated fatty acids), lotions and creams, deodorants, other aqueous or alcoholic solutions, for example cleansing solutions for the skin, moist cleansing tissues, oils or powders.

Accordingly, this invention also relates to body-care products containing at least one compound of formula (1) and to cosmetically compatible carriers or auxiliaries.

The novel body-care product comprises 0.01 to 15% by weight, preferably 0.5 to 10% by weight, based on the total weight of the composition, of the compound of formula (1) as well as cosmetically compatible auxiliaries.

Depending on the body-care product's form of presentation, it contains other components besides the compound of formula (1), for example sequestrants, colourants, perfume oils, thickeners or consistency regulators, emollients, UV absorbers, skin protectives, antioxidants, additives for improving the mechanical properties, such as dicarboxylic acids and/or the Al, Zn, Ca, Mg salts of C₁₄-C₂₂fatty acids, and, optionally, additional antimicrobial active substances.

The novel body-are product may be formulated as water-in-oil or oil-in-water emulsion, as alcoholic or alcohol-containing formulation, as vesicular dispersion of a ionic or non-ionic amphiphilic lipid, as gel, solid stick or as aerosol formulation.

A water-in-oil or oil-in-water emulsion comprising the compound of formula (1) contains as cosmetically compatible auxiliaries preferably 5 to 50% of an oil phase, 5 to 20% of an emulsifier and 30 to 90% of water.

The oil phase can in this case contain any oil suitable for cosmetic formulations, for example one or several hydrocarbon oils, wax, natural oil, silicone oil, fatty acid ester or fatty alcohol. Preferred mono- or polyols are ethanol, isopropanol, propylene glycol, hexylene glycol, glycerol and sorbitol.

Compounds of this invention may be present in different cosmetic formulations. Examples of particularly suitable formulations are the following:

skin-care products, for example skin washing and cleansing products in the form of bars of soap or liquid soaps, syndets or washing pastes,

bath products, for example liquid (foam baths, milks, shower products) or solid bath products, such as bath pearls and bath salts;

skin-care products, such as skin emulsions, multiple emulsions or skin oils;

decorative body-care products, for example face make-ups in the form of day or powder creams, face powders (lose and compressed), rouge or cream make-ups, eye-care products, for example eye shadow products, mascara, eyeliners, eye creams or eye-fix creams; lip-care products, for example lipstick, lip gloss, lip liner, nail-care products, such as nail varnish, nail varnish remover, nail hardeners or cuticle removers;

feminine hygiene products, such as feminine hygiene washing lotions or sprays;

foot-care products, for example foot baths, foot powders, foot creams or foot balms, special deodorants and antiperspirants or products for scrubbing off calluses;

sunscreens, such as sun milks, lotions, creams, oils, sunblockers or tropicals, pre-sun products or after-sun products;

suntanning products, for example self-tanning creams;

depigmenting products, for example products for bleaching or lightening skin;

insect repellents, for example insect oils, lotions, sprays or sticks;

deodorants, for example deodorant sprays, non-aerosol sprays, deodorant gels, sticks or roll-ons;

antiperspirants, for example antiperspirant sticks, creams or roll-ons;

products for cleansing and treating impure skin, for example syndets (solid or liquid), peeling or scrubbing products or peeling masks;

chemical depilatory products, for example depilatory powders, liquid depilatory products, creamy or pasty depilatory products, depilatory gels or aerosol foams;

shaving products, for example shaving soap, foaming shaving creams, non-foaming shaving creams, shaving foams and gels, preshaving products for dry shaving, after-shaves or aftershave lotions;

scents, for example perfumes (Eau de Cologne, Eau de Toilette, Eau de Parfum, Parfum de Toilette, perfume), perfume oils or perfume creams;

products for oral and dental hygiene as well as for dentures, for example toothpastes, tooth gels, tooth powders, mouth-wash concentrates, anti-plaque mouth-washes, denture cleaning products or denture adhesion products;

cosmetic formulations for hair treatment, for example hair washes in the form of shampoos, hair conditioners, hair-care products, for example pretreatment products, hair tonics, hair styling creams and gels, pomades, hair rinses, deep conditioning treatments, intensive hair care treatments, hair setting products, for example waving agents for perms (hot wave, mild wave, cold wave), hair straightening products, liquid hair fixatives, hair foams, hair sprays, bleaching agents, for example hydrogen peroxide solutions, bleaching shampoos, bleaching creams, bleaching powders, bleaching pastes or oils, temporary, semitemporary or permanent hair dyes, products containing self-oxidising dyes, or natural hair dyes, such as henna or camomile.

The composition of an antimicrobial soap is, for example, as follows:

0.01 to 5% by weight of the compound of formula (1),

0.3 to 1% by weight of titanium dioxide,

1 to 10% by weight of stearic acid,

ad 100% of soap base, for example the sodium salts of tallow fatty acid and coconut fatty acid or glycerols.

The composition of a shampoo is, for example, as follows:

0.01 to 5% by weight of the compound of formula (1),

12.0% by weight of sodium-laureth-2-sulfate,

4.0% by weight of cocamidopropylbetaine,

3.0% by weight of NaCl, and

water ad 100%.

The composition of a deodorant is, for example, as follows:

0.01 to 5% by weight of the compound of formula (1),

60% by weight of ethanol,

0.3% by weight of perfume oil, and

water ad 100%.

### EXAMPLE OF O/W EMULSION

(a):

0.01-5% by weight of the compound of formula (1),

12% by weight of glyceryl stearate,

6% by weight of paraffin oil,

6% by weight of caprylic/capric triglyceride,

4% by weight of glycerol,

0.2% by weight of di-sodium EDTA,

1.0% by weight of citric acid (20%), and

65.8-70.8% by weight of water.

(b):

0.01-5% by weight of the compound of formula (1),

3.5% by weight of PEG-30 dipolyhydroxystearate,

10.0% by weight of paraffin oil,

4% by weight of caprylic/capric triglyceride,

4% by weight of dicaprylic ether,

0.2% by weight of di-sodium EDTA,

3.4% by weight of glycerol, and

69.9-74.9% by weight of water.

In another of its aspects, this invention relates to an oral composition, containing 0.01 to 15% by weight, based on the total weight of the composition, of the compound of formula (1) as well as orally compatible auxiliaries.

### EXAMPLE OF AN ORAL COMPOSITION

10% by weight of sorbitol,

10% by weight of glycerol,

15% by weight of ethanol,

15% by weight of propylene glycol,

0.5% by weight of sodium lauryl sulfate,

0.25% by weight of sodium methylcocyl taurate,

0.25% by weight of polyoxypropylene/polyoxyethylene block copolymer,

0.10% by weight of peppermint flavour,

0.1 to 0.5% by weight of a compound of formula (1), and

48.6% by weight of water.

The novel oral composition may be, for example, a gel, paste, cream or an aqueous formulation (mouth-wash).

The novel oral composition can furthermore contain compounds which release fluoride ions which are effective against caries formation, for example inorganic fluoride salts, such as sodium, potassium, ammonium or calcium fluoride, or organic fluoride salts, such as amine fluorides, which are known under the tradename Olafluor.

The compounds of formula (1) used according to this invention are furthermore suitable for the antimicrobial treatment of textile fibre materials. These materials are undyed and dyed or printed fibre materials, for example of silk, wool, polyamide, polyester, polypropylene or poly-urethanes and, preferably, cellulose-containing fibre materials of all kinds. Such fibre materials are, for example, natural cellulose fibres, such as cotton, linen, jute and hemp, and cellulose and regenerated cellulose. Particularly suitable textile fibre materials are those consisting of cotton.

The compounds of formula (1), singly or in combination with other antimicrobial substances, are also suitable for preserving cosmetic products, such as shampoos, bath additives, hair-care products, liquid and solid soaps (based on synthetic surfactants and the salts of saturated and/or unsaturated fatty acids), lotions and creams, deodorants, other aqueous or alcoholic solutions, e.g. cleansing solutions for the skin, moist cleansing tissues, oils or powders, and household products, for example washing and cleaning formulations, such as liquid and powder detergents or softeners.

The compounds of formula (1) used according to this invention are also suitable for the antimicrobial finishing of synthetic materials, for example polyethylene, polypropylene, polyurethane, polyester, polyamide, polycarbonate, latex and the like. Areas of application for these are typically floorings, plastic coatings, plastic container materials and packaging materials; kitchen and bathroom utensils (e.g. brushes, shower curtains; sponges, bathroom mats), latex filter materials (air and water filters), plastic articles which are used in the medical field, for example bandaging materials, syringes, catheters and the like, so-called medical devices, gloves and mattresses.

Paper, for example sanitary papers, can also be antimicrobially finished with the novel compounds of formula (1).

It is also possible to antimicrobially finish nonwovens according to this invention, for example diapers, sanitary towels and pads, tissues for hygienic and household use.

The hydroxyphenyl-1,3-propanediones can, in particular, also be used in household and all-purpose cleaners used for cleaning and disinfecting hard surfaces.

The composition of a cleaning agent is, for example, as follows:

0.01 to 5% of the compound of formula (1),

3.0% of octyl alcohol 4EO,

1.3% of fatty alcohol C₈-C₁₀polyglucoside,

3.0% of isopropanol,

ad 100% of water.

Beside preserving cosmetic and household products, it is also possible to preserve and antimicrobially finish technical products, such as paper treatment liquors, printing thickeners consisting of starch or cellulose derivatives, paint systems and paints.

The compounds of formula (1) are also suitable for the antimicrobial wood treatment and for the antimicrobial treatment and finishing of leather.

The following non-limitative Examples illustrate the invention in more detail.

### EXAMPLE 1

Reaction Step 1a

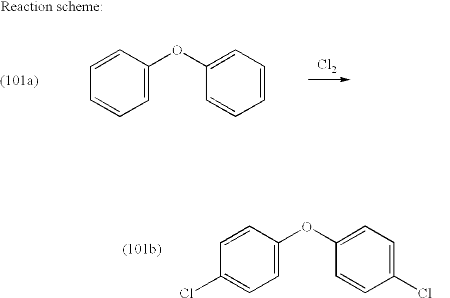

170 g of diphenyl ether are placed in a vessel and are melted, with stirring, at 30 to 40° C. 4.7 g of dipropylsulfide and 4.8 g of aluminium chloride are then added. Chlorination is effected by introducing chlorine at 35 to 40° C. until the reaction mixture has reached the desired degree of chlorination. The reaction is monitored via gas chromatography or via HPLC. The reaction mixture can be separated by distillation and the monochlorodiphenyl ether can be recycled. If chlorination is continued until the di- and trichlorine degree (80% di-chlorine and 20% trichlorine content), the reaction mixture can be used directly for acylation.

### EXAMPLE 2

Acylation (2^{nd} Reaction Step)

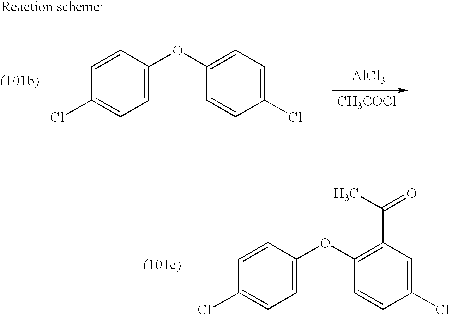

520 g of ethylene chloride are charged, with stirring, with 290 g of aluminium chloride at 20 to 25° C. 170 g of acetyl chloride are added dropwise over 15 to 20 minutes at 25 to 40° C. and the mixture is stirred for 10 minutes at 40° C. Over 1 hour, a solution of 239 g of 4,4′-dichlorodiphenyl ether in 400 g of ethylene chloride is added dropwise to this mixture. The reaction mixture is kept for 4 hours at 40° C. (±2° C.) and is then added to a mixture of 2500 ml of ice water and 350 ml of 34% HCl, thorough stirring being necessary. The phases are separated at 15 to 20° C. The ethylene chloride is removed by distillation from the organic phase.

The residue consisting of crude acyl derivative (=300 g) is dissolved in 1000 g of isopropyl alcohol at 70° C. and clarified by filtration using some activated carbon. With stirring, 350 ml of water are added at 60° C. The solution is slowly cooled, crystallisation being initiated with seeding crystals at 45° C. The solution is stirred for 15 hours and the crystals are collected by filtration at 25° C. and washed with a mixture consisting of 210 g of isopropyl alcohol and 70 ml of water. Drying under vacuum at 50° C. yields 192.5 g of a pale beige and crystalline product of formula (101 c) having a melting point of 65-66° C.

### EXAMPLE 3

Baeyer-Villiger Oxidation

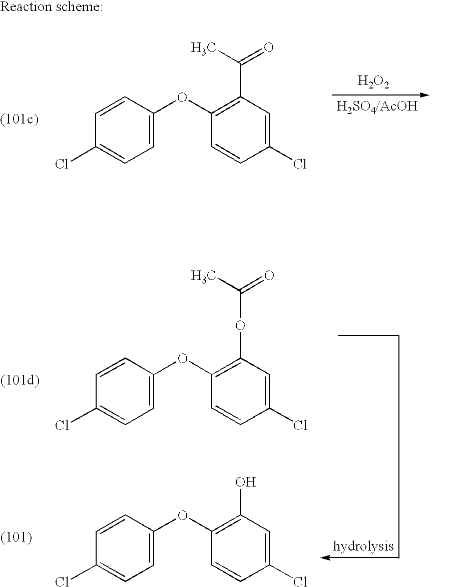

130 g of acetic acid (100%) are placed in a vessel and are mixed, with cooling, with 31 g of H₂SO_{4 conc} at 15 to 20° C. At this temperature, the mixture is charged over 30 minutes with 30 g of hydrogen peroxide (50%). This reaction solution is added dropwise over one hour to a ready solution of 70 g of the acetyl compound of formula (101 c), dissolved in 180 g of acetic acid (100%) and 87 g of H₂SO_{4 con} at 20 to 25° C. The mixture is stirred for 3 hours at 20 to 25° C. and is slowly heated to 45° C. in order to completely hydrolyse the phenol ester of the compound of formula (101 d). This temperature is maintained for 2 hours and the reaction mixture is then added to a mixture consisting of 130 g water and 360 g of toluene so that the final temperature is from 25 to 30° C. The phases are separated, the organic phase is washed with water until neutral and the toluene is removed by distillation. The remaining residue weighs 143 g and consists of the crude product of formula (101). Recrystallisation from petroleum ether 80/110 yields the pure product in the form of colourless crystals having a melting point of 74 to 75° C.

1. A process for the preparation of 4,4′-dihalogen-o-hydroxydiphenyl compounds, which comprises halogenising a diphenyl compound (step 1a) or reacting p-halophenol and p-dihalobenzene in the presence of copper and/or copper salts (step 1b), acylating the di-halogen compound in a Friedel-Crafts reaction (2^{nd} step), oxidising the acyl compound (3^{rd} step) and subsequent hydrolysis (4^{th} step) according to the following reaction scheme: 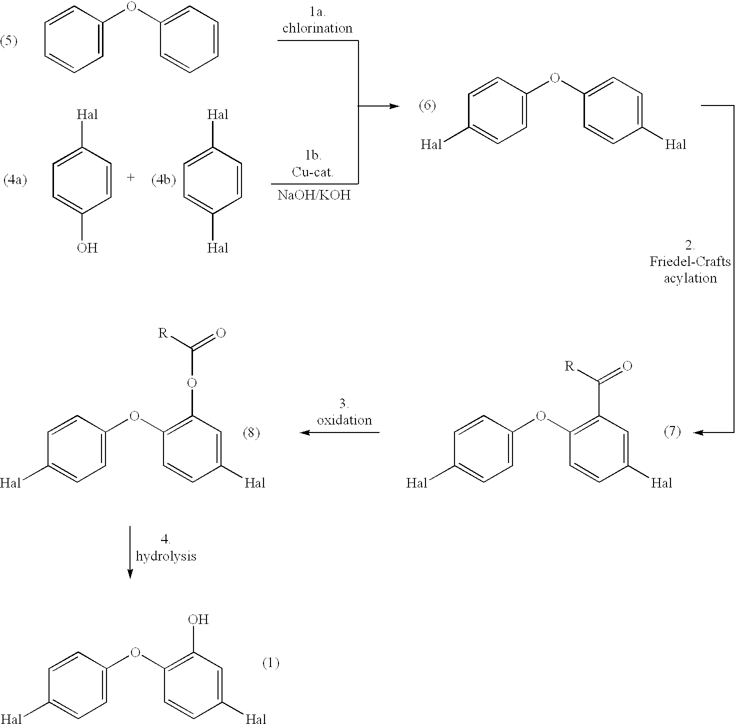
wherein
R is C₁-C₈alkyl which is unsubstituted or substituted by 1 to 3 halogen atoms or hydroxy; unsubstituted C₆-C₁₂aryl; or C₆-C₁₂aryl which is substituted by 1 to 3 halogen atoms, C₁-C₅alkyl or C₁-C₈alkoxy, or their combinations; and
Hal is a halogen atom.

2. A process according to claim 1, wherein
R is C₁-C₄alkyl.

3. A process according to claim 2, wherein
R is methyl.

4. A process according to any one of claims 1 to 3, wherein halogen is chloro.

5. A process according to claim 4, wherein the chlorination (step 1a) is carried out using elementary chlorine.

6. A process according to claim 5, wherein the chlorination is carried out in the presence of a mixture consisting of propyl sulfide and an equimolar amount of aluminium chloride.

7. A process according to any one of claims 1 to 6, wherein the copper catalyst used for the reaction step 1b is copper(II) oxide, copper(I) oxide, copper carbonate, basic copper carbonate, copper(I) chloride, copper(II) chloride, copper(I) bromide, copper(II) bromide or copper sulfate.

8. A process according to any one of claims 1 to 7, wherein the acylation reaction (2^{nd} step) is carried out in the presence of acetyl chloride and aluminium chloride, which are used in equimolar amounts.

9. A process according to claim 8, wherein the acylation reaction is carried out in the presence of a halogenated solvent.

10. A process according to any one of claims 1 to 9, wherein halogenation (first step) and acylation (2^{nd} step) are carried out as a one-pot reaction.

11. A process according to any one of claims 1 to 10, which comprises carrying out the oxidation (3^{rd} step) using a mixture consisting of concentrated sulfuric acid, anhydrous acetic acid and hydrogen peroxide as solvent.

12. A process according to claim 11, wherein the reaction time for the oxidation is from 1 to about 24 hours, the oxidation being carried out at room temperature.

13. Use of the compounds prepared by the process according to any one of claims 1 to 12 for protecting organic materials and objects from attack by microorganisms.

14. Use of the compound of formula (1) for the antimicrobial treatment of the skin, mucosae and hair.

15. Use of the compound of formula (1) for the antimicrobial treatment of textile fibre materials.

16. Use of the compound of formula (1) in washing and cleaning formulations.

17. Use of the compound of formula (1) for the antimicrobial finishing of plastic materials, paper, nonwovens, wood or leather.

18. Use of the compound of formula (1) for preserving cosmetic products.

19. A body-care product, which comprises
0.01 to 15% by weight, based on the total weight of the composition, of the compound of formula (1) as well as cosmetically compatible auxiliaries.

20. An oral composition, which comprises
0.01 to 15% by weight, based on the total weight of the composition, of the compound of formula (1) as well as orally compatible auxiliaries.
